(19) United States
(12) Patent Application Publication
(10) Pub. No.: US 2013/0211254 A1
(43) Pub. Date: Aug. 15, 2013

(54) ULTRASOUND ACQUISITION

(71) Applicants: Svein Arne Aase, Trondheim (NO); Hans Torp, Trondheim (NO); Tore Gruner Bjastad, Trondheim (NO)

(72) Inventors: Svein Arne Aase, Trondheim (NO); Hans Torp, Trondheim (NO); Tore Gruner Bjastad, Trondheim (NO)

(73) Assignee: NORWEGIAN UNIVERSITY OF SCIENCE AND TECHNOLOGY (NTNU), TRONDHEIM (NO)

(21) Appl. No.: 13/700,693

(22) PCT Filed: May 26, 2011
(86) PCT No.: PCT/GB11/50994

§ 371 Date: Feb. 11, 2013

(30) Foreign Application Priority Data
May 28, 2010 (GB) 1009006.6

(51) Int. Cl. A61B 8/08; A61B 8/00

(52) U.S. Cl. CPC A61B 8/085; A61B 8/488; A61B 8/0883; A61B 8/485; A61B 8/461; A61B 8/5223
USPC 600/438; 600/449; 600/450

(57) ABSTRACT

Ultrasound acquisition Information about a structure (701) within a body is acquired by providing data defining a non-straight line (42b), at least a part of which corresponds to the structure (701); transmitting an ultrasound transmit signal (44b) into the body; receiving reflections (400-402) of the transmit signal (44b); processing the received reflections (400-402) so as to trace the focal point (403-405) of a receive beam along the path of the non-straight line (42b); using reflections (400-402) of the transmit signal (44b) from along the non-straight line (42a) to acquire information about the structure (701).

This invention relates to apparatus and methods for acquiring and processing information using ultrasound reflections. It relates particularly, but not exclusively, to medical ultrasonography.

By listening to reflections of ultrasound pulses transmitted into an object, such as a human body, information about the structure and motion of parts of the object can be determined non-invasively. For example, a doctor can use an ultrasound scanner to determine information about the shape and motion of a valve in a patient's heart.

In known arrangements, ultrasound pulses are transmitted into the body in a given direction from a handheld scanning head containing an array of ultrasound transducers. An array of microphones in the scanning head is used to listen selectively to sounds emanating from a particular point or direction in the body.

As a transmitted pulse travels into the body, it creates echoes at progressively greater distances from the scanning head. By measuring the delay between the transmission of a pulse and the receipt of an echo, the distance to the point within the body at which the echo emanated can be determined.

Typically, in order to build up a picture of a 2D slice through the body, a succession of transmit pulses is directed at different angles from the scanning head. For each transmit pulse, receive beam-forming and time-of-flight information are used to determine information about the reflection at different points within the width of the transmit pulse. Receive beam-forming is typically accomplished by having a line of microphones in the scanning head, each connected to a respective delay unit, which can apply a variable time delay to the received signal. The outputs of these delay units are then summed. Since signals from a particular point or direction, relative to the head, will reach each microphone at slightly different times, this knowledge can be used to set the delays such that signals from a desired direction and depth are combined and amplified during the summing operation, while signals from other directions and depths are reduced and filtered out. Further tuning of the receive beam can be achieved by differentially amplifying the signals from each microphone; this is known as apodization.

In 2D scanning, the beam-angle and focal depth can be varied over time in order to acquire sufficient information from a scanning sector to build up a 2D image. A B-mode scan is formed by sweeping a receive beam in a sequence of steps from an extreme angle at one side of the microphone array to an extreme angle at the other side. For each step, a pulse is transmitted which covers the appropriate angle. The transmit pulse may itself be steered by firing an array of transducers at staggered timings. As the transmit pulse moves through the body, it scatters reflections back towards the scanning head. The earlier-received reflections are from points close to the scanning head, while the latter-received reflections are from points further away. The intensity of the signal at any point in time conveys information about the material which scattered the signal at a corresponding distance from the scanning head. The B-mode image is a two-dimensional representation of the sector swept by the receive beam, with the position of the scanning head corresponding to the top of the image, and with lower points on the image corresponding to more distal positions from the scanning head. Pixel intensities represent the intensity of the reflected signal from each point. In this way, a B-mode scan of a human body presents a cross-sectional image through the body, in which different tissue types can be distinguished due to their differing reflectiveness to ultrasound.

The process of acquiring information to create a 2D slice of echo intensities (e.g. for a B-mode scan) is constrained both by the speed of sound in the object and by the processing capabilities of the scanner. When capturing a sequence of 2D slices over time, scan line density and field of view must be balanced against refresh rate. In other words, either the time taken to generate a single B-mode image can be short, allowing for a fast frame rate when producing a sequence of images (e.g. a moving video), or the time taken to generate each image can be longer, allowing for a greater field of view or a higher scan line density. A compromise position must therefore be reached between temporal resolution, field of view, and scan line density, where one can be increased at the expense of one or both of the others. The situation is more pronounced for 3D scans, in which beam-forming is used to scan transmit and/or receive beams through a three-dimensional volume of points. This requires one or more beams to be swept through each of length, breadth and depth axes. For example, expanding a 2D scan with a frame rate of 50 Hz to 3D, while maintaining the same scan line density, can cause the 3D frame rate to drop to only 0.5 Hz (assuming a pulse repetition frequency of 5 kHz). In order to analyse motion within the body, Doppler imaging can be used. This is commonly done in order to monitor the flow of fluid, such as blood, but can also be used to characterise movement of tissue, such as the heart wall. This is known as tissue Doppler imaging (TDI). An extension of TDI is strain imaging, in which motion information for different points can be used to calculate strains within the tissue.

Acquiring accurate Doppler information requires a rapid succession of transmit pulses and echoes from along a scan line. Doppler scanning is therefore often performed along a single, radial scan line. The angle of this line from the scanning head may be set by an operator, based on an earlier B-mode scan of a region of interest. Refresh rates of 1,000 frames per second are possible. However, information is only available from along the scan line. For a substantially flat structure, such as the septal wall, it may, with skill, be possible for an operator to align the scan line along the structure of interest; but for non-flat structures, this is impossible.

It has been proposed in US 2002/0161299 to provide a system for performing arbitrary M-mode ultrasonic imaging in real time. In this document two acquisitions are used. One is a full conventional scan and the other is narrower scan or wedge, which is also acquired as a continuous scan. The actual M-mode is generated in a post processing step by a scan converter on the wedge data.

If Doppler information is required from across a wider scan region, such as for a 2D colour-flow Doppler scan, it is necessary to sweep the beam across this region, resulting in a dramatically lower update rate and/or scan line density.

In addition to phase-array scanning described above, other scanning approaches are also known. For example, switched linear scanning is known, using planar or curved arrays. A sub-array of transmitters is typically used for each transmit signal, with the sub-array being shifted across the surface of the transmitter between successive transmissions. With planar arrays, the transmit beam will typically have a fixed transmission angle but a varying origin, resulting in a rectangular or rhomboidal scan sector. With a curved array surface, both the angle and the origin will typically change between successive transmit beams.

Pixel-based receive beamforming is an alternative to conventional scan line receive beamforming. The transmit beam grid is identical for the two approaches, but instead of carrying out receive beamforming along radial lines, beamforming is carried out along vertical or horizontal lines in a rectangular grid. These lines may then extend along one or more transmit beams.

However none of these alternative arrangements overcomes the temporal resolution problem identified above.

An attempt to address the problem is presented in WO 2004/051310, which discloses an ultrasound system that automatically identifies “regions of interest”, being those regions that include tissue motion or blood flow. Once these flow-containing regions have been automatically identified and distinguished from non-flow regions, analysis such as colour-flow Doppler or spectral Doppler, using a swept beam, is applied just to these regions.

However, such an approach is not always ideal. In particular, the regions identified automatically by the system as containing flow will not necessarily correspond exactly with the area or areas of actual interest to a human operator. Resources may therefore still be wasted in obtaining information from regions which are not of clinical interest. This can result in inferior temporal resolution, which may be especially problematic during real-time analysis.

The present invention seeks to address such shortcomings.

From a first aspect, the invention provides a method of acquiring information about a structure within a body, comprising:
providing data defining a non-straight line, at least a part of which corresponds to the structure;
transmitting an ultrasound transmit signal into the body;
receiving reflections of the transmit signal;
processing the received reflections so as to trace the focal point of a receive beam along the path of the non-straight line; and
using reflections of the transmit signal from along the non-straight line to acquire information about the structure.

The invention extends to ultrasound scanning apparatus for acquiring information about a structure within a body, comprising:
means for providing data defining a non-straight line, at least a part of which corresponds to the structure;
transmitting means arranged to transmit an ultrasound transmit signal into the body;
receiving means arranged to receive reflections of the transmit signal; and
processing means configured to:
   process the received reflections so as to trace the focal point of a receive beam along the path of the non-straight line; and
   use reflections of the transmit signal from along the non-straight line to acquire information about the structure.

The invention further extends to software, and a carrier bearing software, comprising instructions for configuring a processor to carry out the steps of:
processing received signals from reflections of an ultrasound transmit signal transmitted into the body, so as to trace the focal point of a receive beam along the path of a non-straight line, at least a part of which corresponds to a structure within a body; and
using reflections of the transmit signal from along the non-straight line to acquire information about the structure.

Thus it will be seen by those skilled in the art that, in accordance with the invention, ultrasound scanning can be performed by tracing a receive beam along a non-straight, or curved, path, following the shape of a physical structure of interest, such as the heart wall. The invention therefore overcomes the need to waste limited resources scanning a larger sector (2D) or volume (3D), using straight scan lines, to ensure coverage of the part of the structure that is of interest to the clinician.

Rather, the receive beam can be concentrated on precisely the areas of interest. This is more computationally efficient and enables a much higher temporal update rate to be obtained, because the number of transmit beams need not be as great. Computational efficiency can be very important, since processing resources will typically be constrained, for example, because the scanning apparatus is a portable device, or due to cost considerations. Where the processing steps happen in real-time or near real-time, a more efficient allocation of processing resources can enable substantially higher update rates than would otherwise be possible, especially when scanning in 3D.

It has previously been proposed to use curved receive beams in ultrasound scanning for other purposes such as to compensate for geometrical distortions which can arise when using parallel beamforming in swept scans. However, such receive beams do not correspond to a structure in the body and do not address the problem of increasing temporal update rates or computational efficiency.

The receiving means preferably comprises an array of receiver elements, such as a linear array (which may be straight or curved) or a phased array. These may be contained in a scanning head or probe.

Those skilled in the art will appreciate that various approaches can be used to trace the focal point of a receive beam along the path of the non-straight line. The tracing may be accomplished in real-time (e.g. as the reflections are received, or at least before the next transmit pulse is sent) or in a post-processing phase (e.g. after one or more subsequent transmit pulses has been sent). It will be appreciated however that embodiments of the invention can avoid the need for post-processing scan conversion as is necessary in some prior art proposals. Receive beams may be steered in real-time or constructed afterwards using delay-and-sum beamforming techniques, either in hardware or software. The processing means may comprise or consist of hardware and/or software logic.

The data defining the curved line may be provided in any appropriate way. In one set of embodiments, data defining the non-straight line are input from a human operator via input means, such as a mouse, trackball, touchpad, keyboard or touch-screen display. The means for providing data may comprise or consist of logic, e.g. in software and/or hardware. The means may comprise a communications link, such as a wire or radio interface. It may alternatively or additionally comprise a processor and/or peripherals, such as a display screen, keyboard, mouse, etc.

The provided data could simply comprise a sequence of coordinates of individual points. These coordinates may be defined with reference to any suitable origin, for example a point on the face of a scanning head. In other embodiments, the data comprise one or more parameters which together uniquely determine the non-straight line. The non-straight line may be defined by a predetermined equation or curve-fitting algorithm, taking these parameters as inputs. In some embodiments, the parameters are spatial coordinates. The non-straight or curved line may be smoothly curving, i.e. everywhere differentiable, but this is not essential. It may, for example, include non-differentiable points, such as corners or discontinuities.

In some embodiments, the non-straight line is defined by an equation, e.g. a polynomial equation, in n variables, and the provided data comprise values for these variables. In a yet further embodiment, the curved line is defined by a regression algorithm as a best-fit to a set of received coordinates.

Preferably the method comprises displaying an image of at least a part of the structure, to enable the line to be defined with reference to that image, e.g. by a human operator. For example, a B-mode scan, which may be moving or static, may be displayed on a monitor, and a human operator may identify points on the scan through or near which the non-straight line should pass, for example by moving a cursor to each point in turn and clicking a button, by touching a touchscreen display at the relevant point or by dragging a flexible candidate line. A representation of the non-straight line may be displayed on a display screen, for example, overlying an image of the structure. Adjustments may be made using input means, for example, by clicking and dragging with a cursor to reposition part or all of the line.

As an alternative to displaying the curved line on a display surface, an estimate of the actual area or volume from which data will be collected may be visualised. This might appear as a line with thickness, which follows the same route as the curved line would. Adding thickness to the line allows the actual acquisition region to be indicated. Alternatively, a colour gradient could be overlaid on an image, in which a first colour represents a predominant acquisition region (close to where the curved line might otherwise lie) and in which one or more further colours represents surrounding regions having lower acquisition sensitivities. Gradients based on other features such as shading could be used instead of colour. Such an estimate of the actual area or volume may be based on calculations or simulations of the transmit and/or receive beam(s). One approach for representing the sensitivity of an ultrasound beam visually, using simulation, is described in Bjastad, T.; Aase, S. A.; Torp, H.; “Velocity sensitivity mapping in tissue Doppler images”, Ultrasonics Symposium, 2005 IEEE, vol. 4, pp. 1968-1971, 18-21 Sep. 2005.

In another set of embodiments, the data defining the non-straight line are provided by means of a theoretical, e.g. mathematical model of the structure. The model may be of predetermined, fixed size; in this case it may be selected based on criteria provided by the operator, such as the age, height and/or weight of the patient. In other embodiments, a model including variable parameters is fitted to the structure using data acquired by ultrasound scanning, and the non-straight line is defined using information from the model. Combinations of, and variations on, these two approaches may be used. One approach to modelling a structure, which may be used here, is described in US 2008/0069436.

In an exemplary clinical setting, imaging data relating to the left ventricle of the heart are acquired, for example using relatively low-resolution 2D or 3D scanning, and these data are used to calibrate a deformable model of the left ventricle. Data defining a desired, curved imaging line are extracted from the model, and high-resolution data acquisition is performed along the curved line.

The model parameters may be determined to correspond to the structure without substantive human involvement (fully automatic operation), or a human operator may assist in fitting the model to the structure (semi-automatic operation), for example, by viewing a B-mode scan of the structure and an overlaid mesh outline of the model on a display screen, and providing instructions to align the model e.g. by translating, rotating, scaling, stretching, or otherwise deforming it, in order to achieve a satisfactory correspondence between the model and the structure. Using a model to determine the non-straight line can avoid a human user having to define the line manually, thereby relieving the user of this task. The line may, in some circumstances, more closely correspond to the structure as a result, since, for example, a greater number of parameters may be determined, which would be unduly burdensome if a human operator were determining the line. Furthermore, the potential for human error is removed. Nonetheless, greater computational resources will typically be required, and manual determination may be preferable in some situations. In some embodiments, both manual and automatic data sources may be supported, e.g. in alternative modes of operation.

A repetitive process of acquiring updated imaging data, recalibrating the model accordingly, and acquiring updates along the curved line may be carried out. If the non-straight line moves, this may be shown on a display screen, overlying a static or moving image of the scanned region.

In some embodiments, a human operator provides input defining boundaries within which the non-straight line must lie, thereby constraining the model but allowing for movement of the structure.

In a further set of embodiments, the non-straight line is determined directly, e.g. by the processing means or another processor, applying a criterion or filter to data obtained from ultrasound reflections from the structure. A prior model of the structure is not necessary. For example, a processor may define the non-straight line by analysing an imaging scan to identify a boundary between two tissues and by defining the line so as to lie along this boundary. Where the scan is planar (2D), the line could lie in the scan plane, along the boundary. Where the scan is 3D, some additional constraint may be provided by the user, or may be predefined, to specify how the line should lie along the boundary surface. One or more image-processing algorithms may be implemented on the processor in order to derive information about the structure from an image; from this information, the line may be defined which corresponds to the structure. A human operator may guide this process, e.g. by defining a sub-region of the image, containing a tissue boundary; or it may require no human input.

Irrespective of how the non-straight line is initially defined, it may need to be updated in order to continue corresponding to the underlying structure, when the structure moves relative to a scanning head (for example, because the structure is moving within the body, or because the operator's hand does not hold the scanning head steady). Such updating may occur between every signal transmission, or less frequently, depending on available processing resources and the rate at which the structure is changing.

The non-straight line may correspond to the structure in any way a clinician deems appropriate, when seeking to acquire information about that structure. The correspondence may, for example, be that the non-straight line lies wholly or substantially within or adjacent the structure, or has more than a predetermined proportion of its length (e.g. 75%, 50% or 25%) within or adjacent the structure. Alternatively or additionally, the non-straight line may lie along, or spaced away from, a boundary surface between the structure and an adjacent region; for example, it might lie on the outer surface of the heart, or within a wall of the heart. The distance between the structure and the line may be less than a predetermined maximum value for all points along some or all of the line. In a further set of embodiments, the line may follow, or lie parallel to, the path of a curved, elongate structure (e.g. a blood vessel). In general, the non-straight line may lie in a plane (2D scanning) or may be three-dimensional (3D scanning).

In some embodiments, the non-straight line is parameterised and the values of the parameters are optimal according to a predetermined metric. For example, the values may be such that the line lies within or along the structure to a greater extent than a line produced by any other selection of parameter values would. Alternative criteria may be less strict. The measure of fit may be defined in any appropriate way.

The body could be any object but is preferably a human or animal body.

The structure imaged is preferably subcutaneous. It may comprise an organ, such as the heart, or part of an organ, such as heart valve. The structure may be a contiguous region occupied by a single tissue type, such as layer of fat.

One advantageous implementation or application of the invention which is envisaged is one in which the structure is the outer or lateral wall of the left ventricle. Since this wall is not planar it can be scanned more efficiently in accordance with the present invention than by prior art methods. Other structures which are suited to scanning in accordance with the present invention include blood vessels, such as the carotid artery.

The focal point of a receive beam is a point or region for which a relatively-high or maximum amplification is applied to signals emanating therefrom, when compared with the amplification applied to signals received from other points or regions. It may cover a region of any appropriate size, which may vary between near-field and far-field sources. Beam-forming delay values, apodization functions and/or time-of-flight information may be used to define and control the focal point.

When processing in real time, the focal point is preferably traced, or steered, along the line at approximately half the speed of sound in the body; i.e. such that the focal point travels along the non-straight line in synchronisation with the echoes from the transmit signal. This speed is approximate to account for the curves in the line. The component of velocity in a radial direction from the scanning probe is preferably equal to half the speed of sound.

The transmit signal is preferably a pulse. It may be focussed, unfocussed, or partially focussed. In some embodiments it is a plane wave, which may be transmitted through a relatively wide aperture. If the transmit signal is a plane wave, this wave may be steered at an angle chosen so as to direct energy preferentially towards the structure.

In preferred embodiments, energy from the transmit signal is directed so as to encompass all of that part of the curved line which corresponds to the structure. A plane wave is well suited to this, although other approaches can be used, such as a defocused beam, a slightly focused beam, a small transmit aperture, a limited diffraction beam, or multiple transmission lines.

In some embodiments, the ultrasound transmit signal may be transmitted into the body before data defining a non-straight line have been provided. For example, the received reflections may be recorded as raw signals and only later processed to trace a focal point of a receive beam along a non-straight line.

When the definition of the non-straight line is only provided after the transmission, e.g. in a post-processing step which may happen several hours after the reflected signals have been received, it is not possible to use knowledge of the non-straight line in order to direct the energy from the transmit signal so as to encompass all of that part of the line which corresponds to the structure. In some embodiments, therefore, data are provided that define a transmit path, and energy from the transmit signal travels at least along this transmit path. The transmit path may be defined by any of the steps described herein with reference to the non-straight line; for example, by an operator defining the transmit path on a display screen, or using a model of the structure. In this way, it is possible to acquire reflections from regions through which it is likely that a non-straight line may later be defined, without necessarily needing to receive reflections from across an entire scanning sector. In this way, advantages of a faster update rate can still be achieved when using a post-processing approach, since it is not necessary to saturate a large area with transmit beams.

Certain embodiments of the present invention are particularly advantageous when used with Doppler scanning, such as fluid or tissue Doppler scanning. In the prior art, Doppler scanning is performed along one or more straight lines emanating radially from the scanning head. Because a faster refresh rate is typically desired for Doppler scanning than for basic imaging, and because several transmit pulses must be sent along the same path to obtain the Doppler information, the limitations of known approaches become even more apparent in this situation. Rather than having to use several Doppler scan lines in order to acquire motion information about a region of interest, or not being able to acquire useful information at all, the present invention allows a single, non-straight scan line to be used to obtain relevant information about the structure.

An example which highlights this advantage is acquiring strain or strain rate information e.g. within the heart muscle tissue of the left ventricle. Such information is calculated using data on the motion of different points through the tissue region of interest. For example, if a point is moving towards the scanning head faster than an adjacent, more-distal point, it can be inferred that the tissue is stretching between these points.

Using tissue Doppler imaging (TDI) velocities can be calculated from the phase shift between two transmit pulse firings in the same direction. If this phase shift is larger than 180°, the velocity cannot be determined unambiguously. The time between two firings in the same direction is known as the pulse repetition time (PRT). The pulse repetition frequency (PRF) is 1/PRT. The relation between the PRF and the maximum velocity that can be unambiguously identified (the Nyquist velocity, V_{nyq}) is: PRF=4*V_{nyq}*f₀/c, where f₀ is the receive centre frequency and c is the speed of sound in the body. Typical tissue velocities encountered within the heart are up to 16 cm/s. Thus the PRF must be approximately 1 kHz to determine tissue velocities accurately.

With known, swept acquisition, the time between each firing in a single direction is relatively long. Typically, for B-mode imaging, the maximum frame rate achievable is below 100 frames per second. Such a low update frequency cannot be used for tissue Doppler imaging. To address this problem, prior art scanners typically use packet acquisition. With packet acquisition each frame is split up into groups of one or more transmit beams. Each group is scanned two or more times before proceeding to the next group. The pulse repetition frequency is then less constrained; however, a problem with this approach is that a longer acquisition time is needed to build up a complete frame.

However, by placing a non-straight scanning line within and around the wall of the heart, in accordance with the present invention, much greater frame rates are possible; for example, 1000 frames per second. This enables strain information within the heart wall to be determined with greater temporal resolution.

This also removes the need to use packet acquisition, since only a few transmit beams need be used and the phase shifts can be calculated on a frame-to-frame basis. This enables a substantial increase (e.g. a doubling or more) in the frame rate compared with packet acquisition and allows for more sophisticated Doppler processing due to a greater number of available samples.

The same approach can be used for other forms of deformation imaging, including pulse wave Doppler spectrums, M-modes and colour M-modes.

In one set of embodiments, triggered acquisition is used in which an imaging (e.g. B-mode) scan is performed during one heartbeat cycle, and then one or more subsequent heartbeats is/are used for acquiring data along one or more non-straight lines. Averaging of the data across a plurality of heartbeats may be used to remove noise. However, in some preferred embodiments, averaging is not required since the invention allows sufficient accuracy for real-time presentation of results.

The benefits which can be achieved in some embodiments of the invention over known approaches are even greater when the scan line is not planar, i.e. when it curves in three dimensions. In order to provide comparable coverage to that obtained by such a non-planar line, the prior art would require a 3D scanner to sweep a beam both lengthways and widthways across a scanning pyramid, perhaps at a refresh rate of only 8 frames per second. Embodiments of the present invention, by contrast, can still achieve similar refresh rates to those achieved by a non-straight line lying in a plane.

In some circumstances, it may be advantageous to scan along a plurality of non-straight lines, which is readily accomplished by embodiments of the present invention. The scan lines may correspond to the same part of the same structure, or to different parts of the same structure, or to different structures. The plurality of lines may be coplanar, such as when performing 2D scanning, which could, for example, allow motion information to be acquired simultaneously from both left and right sides of the heart's external walls (with one scan line corresponding to each wall).

Other benefits can be obtained, however, when the scan lines are not co-planar. This allows a complex three-dimensional structure to be scanned efficiently. For example, it enables a set of M-mode intensity scans, or of Doppler motion or strain scans, to be obtained substantially simultaneously, in real-time around the walls of the left ventricle. A plurality of transmit signals may be used, each covering a different part of the structure, for example, eight transmit beams may be evenly spaced in a cone-shaped arrangement. Two or more transmit pulses may be transmitted simultaneously, but preferably they are transmitted at staggered intervals, to avoid interference. Each transmit beam may relate to one non-straight receive beam or multiple such beams. Each receive scan line may individually lie in a plane or not.

The high temporal resolution which can be achieved in accordance with preferred embodiments of the invention can be used for tracking shear waves in tissues. Properties such as the stiffness of the tissue can be determined from shear wave velocities. In one set of exemplary embodiments, a non-straight line is defined which corresponds with a wall of the left ventricle of the human or animal heart, and reflections of the transmit signal from along this line are used to acquire information about the propagation of a shear wave, for example a shear wave caused by the aortic valve of the heart closing. In some such embodiments, two non-straight lines are defined to correspond respectively to the septal wall of the left ventricle and to any other wall of the left ventricle, and ultrasound reflections from along these lines are used to determine information relating to a time delay between an event related to the closing of the aortic value detected from the basal septum and an event related to the closing of the aortic value detected from another location. One transmit beam may be directed along the septal wall, and a second transmit beam along another wall, such as the lateral or outer wall. The scanning may be two-dimensional, i.e. with two co-planar non-straight received lines; or there may be a plurality of non-straight receive lines for one or both of the walls.

However the invention is used, data acquired along a non-straight line may be presented on a display, such as a monitor or a paper printout. Intensity information over time may be presented using an M-mode type representation, where points along the distance axis represent positions around some or all of the non-straight line. Motion and strain information along the curved line may be presented similarly, or in any appropriate manner. Colour may be used, as will be appreciated by the skilled person.

In some preferred embodiments, information acquired along the non-straight line is alternated or superimposed with intensity information in the form of an image. In one set of embodiments, a 3D perspective representation of the structure is displayed on a surface, with colour used to show strain data acquired along one or more non-straight lines around or near the surface of the structure. A series of such 3D models through time may be presented sequentially.

The inventors have appreciated that reproducing an ultrasonic scan from along a non-straight line is new and inventive in its own right, and thus, from a second aspect, the invention provides a method of processing ultrasonic reflections from within an object, comprising:
providing data defining a plurality of non-straight lines lying at least partly within the object;
receiving data relating to ultrasonic reflections from within the object;
creating a first data set by processing the received data to determine, for each of the lines, a respective value of a property of the object at each of a plurality of points of the object lying along the line;
creating a second data set by processing data relating to subsequently-received ultrasonic reflections to determine updated values of the property for said points of the object; and
graphically representing said first and second data sets for at least some of said points.

The invention extends to ultrasound scanning apparatus comprising:
means for providing data defining a plurality of non-straight lines lying at least partly within the object;
receiving means arranged to receive data relating to ultrasonic reflections from within the object;
graphical display means; and
processing means configured to:
   create a first data set by processing the received data to determine, for each of the lines, a respective value of a property of the object at each of a plurality of points of the object lying along the line;
   create a second data set by processing data relating to subsequently-received ultrasonic reflections to determine updated values of the property for said points of the object; and
   control the graphical display means so as to cause said first and second data sets to be represented graphically for at least some of said points.

The invention further extends to software, and a carrier bearing software, comprising instructions for configuring a processor to carry out the steps of:
creating a first data set by processing data relating to ultrasonic reflections from within an object to determine, for each of a plurality of non-straight lines lying at least partly within the object, a respective value of a property of the object at each of a plurality of points of the object lying along the line;
creating a second data set by processing data relating to subsequently-received ultrasonic reflections to determine updated values of the property for said points of the object; and
controlling graphical display means so as to cause said first and second data sets to be represented graphically for at least some of said points.

This aspect of the invention shares some of the advantages already presented with reference to the first aspect. In particular, by determining values along a plurality of non-straight lines, relevant information can be determined and displayed at a higher temporal and/or spatial resolution than would be possible if indiscriminate scanning of the object were performed using straight scan lines. Computational resources can be employed far more efficiently.

In some preferred embodiments, the non-straight lines do not all lie in a common plane. The use of non-straight lines in such a 3D-scanning situation can be particular advantageous, as it can allow the display screen to show a greater proportion of relevant information than might typically be the case if conventional volumetric scanning were used.

The 3D model may be flattened, sectioned or traversed by a line to display the information of the 3D model as a 2D surface representation.

In some embodiments the provision of data defining the plurality of non-straight lines may be carried out in the ways explained previously (e.g. manually, semi-automatically or fully automatically). More generally, features of the first aspect of the invention may also be features of the second aspect, and vice versa.

The values may represent the intensity of the reflect signal at, or within a predetermined radius of, each point. Alternatively the values may represent the absolute or relative speeds of parts of the object. In some preferred embodiments each value represents the magnitude of the component of the velocity of a part of the object in a particular direction; for example, in the direction of the receiver elements. Alternatively still, the values may represent strain (typically a component of strain along a radial axis), or any other physical attribute of the object at each point.

Because the updated values are determined for the same points of the object, this means that, where the object is moving or changing shape, the updated values correspond to the same physical parts of the object. In practice, this may require the non-straight lines to be morphed in real time, to follow movements of the object. This may be accomplished using modelling or other tracking techniques, for example as discussed above. It will be appreciated, however, that the points of the object to which the first data set relates, and the points to which the second data set relates, may not always be exactly identical, due to limitations in the modelling or tracking process and to deformation of the object; therefore a margin of error may be provided for. The points need not be precise points, but may be strictly or loosely defined regions within the object.

Information from the first and second data sets may be presented simultaneously; e.g. as a plurality of M-mode scans shown on a display surface at the same time. Alternatively, it may be presented separately in time; e.g. as different time frames of one or more moving video sequences.

In some embodiments, the processing is conducted in real time; i.e. within the duration of one transmit cycle, or at least within the duration of a small integer number of transmit cycles, e.g. less than 100, or preferably less than 10. One or more transmit signals may be transmitted into the object, and reflections may be received at receiving means. The data relating to the reflections may be provided in real-time or near real-time; e.g. substantially simultaneously with the display of the first and/or second data sets, for example within the same transmit cycle. However, this is not essential, and received reflections may be stored and subsequently analysed (for example, after ceasing transmitting any further transmit signals) in order to determine, for each of the points along each of the curved lines, the value of a property of the object at that point and then to represent values graphically on a display. In this case, the data relating to the reflections may be received from data storage means, such as a magnetic or silicon memory device. For example, a technician may scan a patient, and a physician may later (possibly an hour or more later) define non-straight lines, with the values along those lines then being determined by analysis of the stored data.

Certain preferred embodiments of the invention will now be described, by way of example only, with reference to the accompanying drawings, in which:

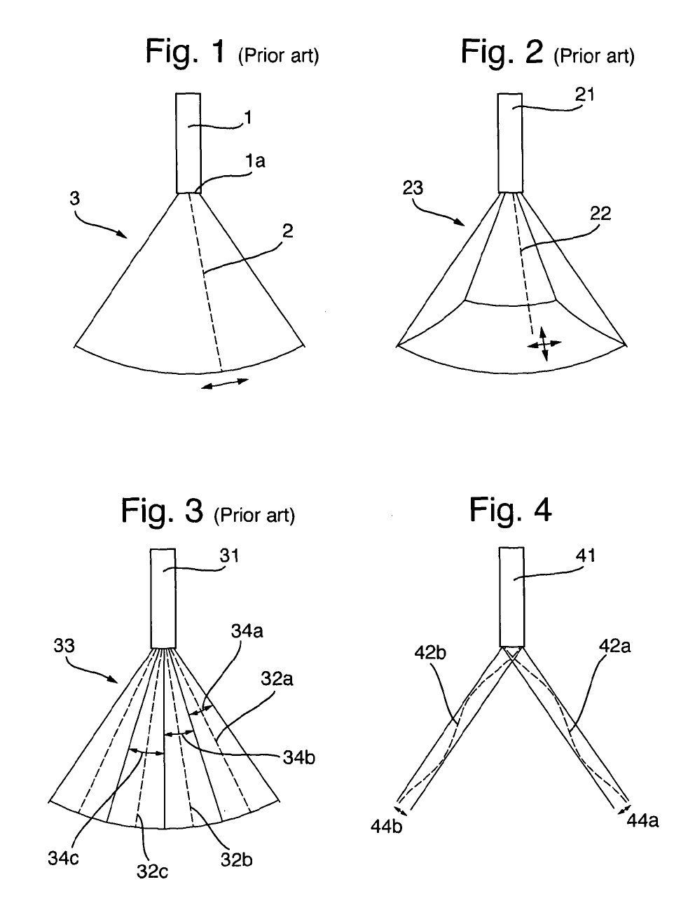

FIG. 1 is a schematic side view illustrating a known 2D ultrasound scanning principle;

FIG. 2 is a schematic side view illustrating a known 3D ultrasound scanning principle;

FIG. 3 is a schematic side view illustrating a known ultrasound scanning principle using multiple transmit beams;

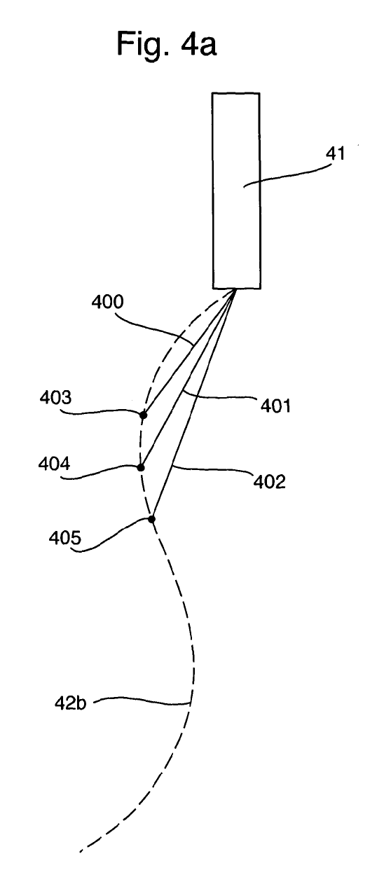

FIG. 4 is a schematic side view illustrating a use of 2D curved receive beams in accordance with the invention;

FIG. 4a is a schematic side view showing one of the receive beams in more detail;

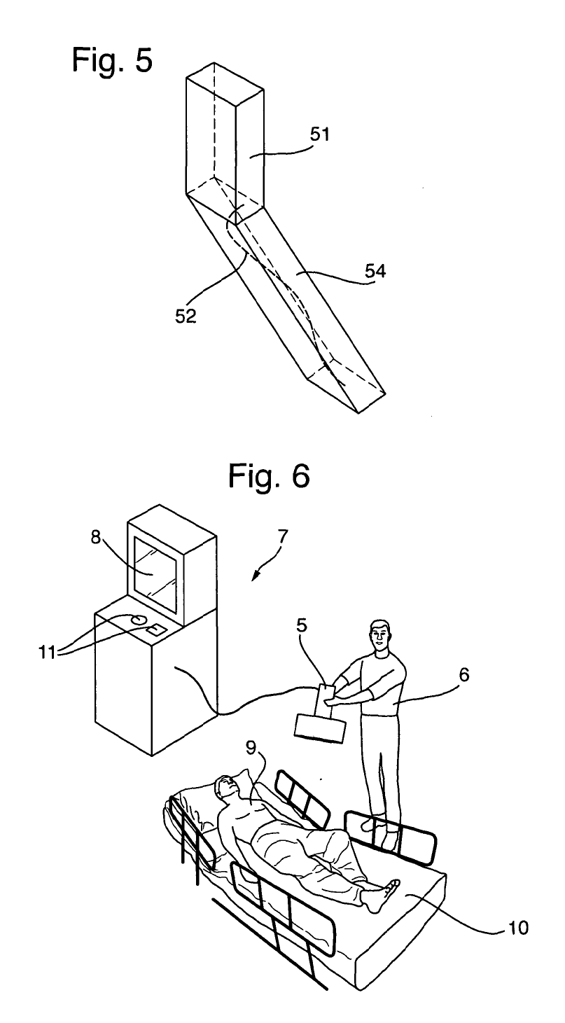

FIG. 5 is a schematic perspective view illustrating a use of 3D curved receive beams in accordance with the invention;

FIG. 6 is figurative perspective drawing of a scanning system in accordance with the invention;

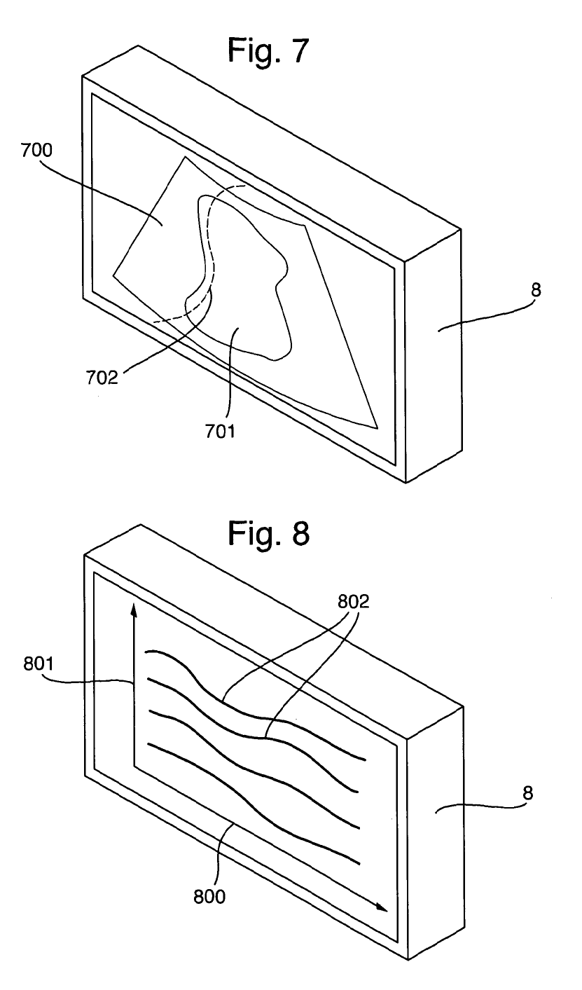

FIG. 7 is a perspective drawing of a display screen when the scanning system is operating in a first mode;

FIG. 8 is a perspective drawing of a display screen when the scanning system is operating in a second mode;

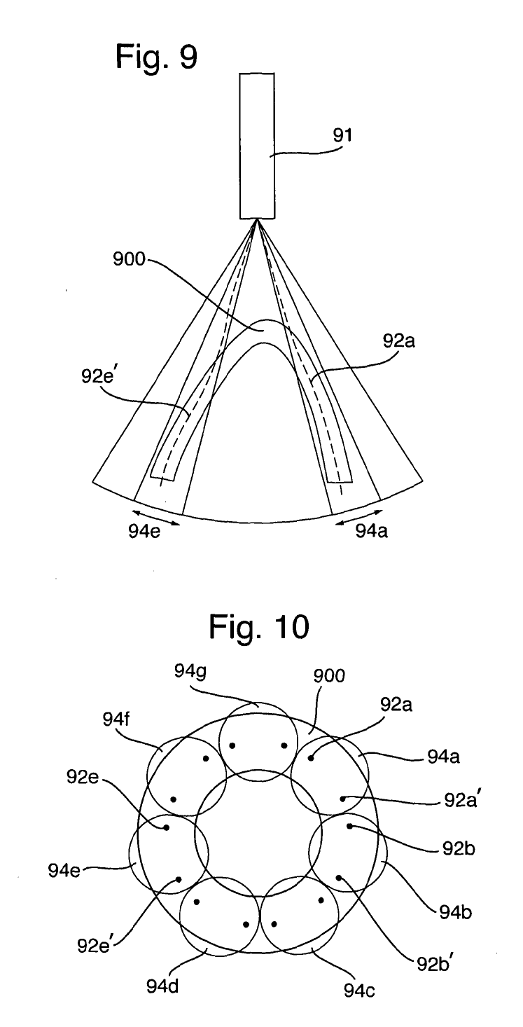

FIG. 9 is a schematic side view illustrating 3D transmit beams in a method in accordance with the invention; and

FIG. 10 is a schematic plan view illustrating the 3D transmit beams.

FIG. 1 shows a 2D ultrasound scanning probe 1 being controlled by a conventional scanning system. It has a front face 1a containing a transmitter from which ultrasound signals are transmitted, and a linear array of microphones at which reflections of these signals are received. In use, an ultrasound pulse is transmitted from the face 1a of the probe into the body of a patient, and the signals received at some or all of the microphones are summed after respective delays have been introduced in the signal path for each microphone. This has the effect of amplifying reflections from along a virtual receive beam 2, while suppressing reflections from elsewhere. By setting the delays appropriately, the angle of the receive beam 2 can be changed for each subsequent transmit pulse. By measuring the time each part of the reflection is received at the array for a given receive beam, the depth of each part of the reflected signal along the receive beam 2 can be determined. By transmitting repeated pulses and sweeping the receive beam 2 from one side to another in an arc, information about the material within the scanning plane 3 can be determined.

FIG. 2 shows a known 3D ultrasound scanning probe 21 being controlled by a conventional scanning system. Rather than a linear array of microphones, this probe contains a two-dimensional array of microphones. By applying appropriate delays to the signal from each microphone, the angle of the receive beam 22 can be determined in two orthogonal axes. By sending a series of transmit pulses and varying both receive-beam angles, the receive beam 22 can be methodically swept through a three-dimensional, pyramid-conical volume 23. Typically, the beam 22 is swept from left to right for a first, constant angle with the plane of the drawing, and is then swept from left to right for a second, constant angle with the plane of the drawing, and so on. It can therefore take many times longer to scan a 3D volume 23 than a 2D sector 3.

FIG. 3 shows a known 2D ultrasound scanning probe 31 which has a plurality of ultrasound transmitters, allowing a pulse of ultrasound to be emitted at a desired angle within the scanning plane 33, under the control of a conventional scanning system. Transmit beams 34a, 34b, 34c can be emitted sequentially or simultaneously at different angles, with respective virtual receive beams 32a, 32b, 32c being used to listen for the reflections.

FIG. 4 shows a 2D ultrasound scanning probe 41 under the control of a scanning system embodying the invention. Two transmit beams 44a, 44b are transmitted, either sequentially or simultaneously, as plane waves at different angles. By adjusting the delays applied to the signals from some or all of the microphones in the probe 41 during the period over which the reflections are received, two respective receive beams 42a, 42b can be made to follow a curved path within the area illuminated by the transmit beams 44a, 44b. As explained below, these curved paths are arranged to correspond to the shape of a structure within the patient's body.

FIG. 4a shows how a receive beam can be made to follow a curved path 42b. Shortly after the transmit pulse 44a has been transmitted, the respective delays on the array of receiver elements in the scanning probe 41 are set so as to cause echoes that have travelled along a path 400 from a proximal point 403 on the curved path 42b to be summed together across the receiver elements, thereby preferentially amplifying signals from the point 403 and filtering out noise from other areas. A short while later, as the transmit pulse moves further away from the scanning head 41, the delays applied to the receiver elements are changed such that echoes travelling along a path 401 from a more distal point 404 on the curved line 42b are summed together. Later still, the delays are altered so that echoes from a yet more distal point 405 along the curved path are preferentially amplified. In order to receive these echoes, the timing of the delay adjustments must be such that a focal point is traced along the curved path 42b in synchronisation with the time taken for the wave front of the transmit pulse to propagate out from the scanning probe 41 and for the echoes to return to the probe.

FIG. 5 shows a 3D ultrasound scanning probe 51 under the control of a scanning system embodying the invention. This probe 51 is able to direct a transmit pulse in a desired direction, in order to illuminate a region of interest with an ultrasound beam 54. By adjusting the delays applied to some or all of the microphones in a two-dimensional array of microphones in the probe 51, a receive beam can be made to follow a non-planar curved path 52, within the region illuminated by the transmit beam 54.

FIG. 6 shows an ultrasound probe 5 being held by a human operator 6, such as a doctor. The probe is connected to a scanning machine 7, which includes a display screen 8. The probe 5 can be applied to the body of a patient 9, here shown lying on a bed 10, in order to obtain information, such as images, of a structure inside the patient 9. For example, the operator 6 may wish to view the womb, or to perform tissue Doppler imaging of the heart. The scanning machine 7 has input controls 11, such as a trackball and keyboard, and is configured to allow the operator 6 to adjust parameters of the scan by means of the input controls 11.

FIG. 7 shows the display screen 8 of the scanning machine 7. It is displaying a representation of a 2D scanning plane 700, and a corresponding B-mode cross-section showing an image 701 of a structure such as the patient's heart. Also shown on the display screen is a representation of a curved line 702, which follows part of the perimeter outline of the structure 701.

The curved line 702 can be positioned in various ways, depending on the exact implementation. In one mode of operation, an operator can add, remove, select and adjust the position of points along the path of this curved line 702 by manipulating the controls 11 of the scanning machine 7. The scanning machine 7 generates an appropriate line fitting these points, based on a built-in curve-fitting algorithm, and presents a visual representation of the line 702, overlying the B-mode image. In the way the operator can use the display 8 to align the curved line 702 to ensure a good correspondence with the structure.

In an alternative mode of operation, the scanning machine 7 automatically determines information about the structure from the B-mode scan, for example, using edge detection, or model fitting, and positions the curved line according in a predetermined alignment.

When scanning, the scanning machine 7 applies appropriate delay-and-sum values, aperture sizes, and apodization functions in order to steer an ultrasound receive beam along the path represented by the curved line 702 on the display screen 8.

Information acquired from along the curved receive-beam path can be used in a variety of ways.

FIG. 8 shows the display screen 8 showing an M-mode scan, with a horizontal time axis 800 and a vertical depth axis 801. Each vertical column represents positions along the curved path. Patterns 802 in the M-mode scan provide information on the movement of those parts of the structure which lie along the path.

FIG. 9 shows a transverse-planar view of a 3D ultrasound probe 91 and a transverse cross-section plane 93 through the left ventricle 900 of a human patient. The boundaries on the plane 93 indicate the extend of the scanning sector provided by the probe 91. The probe 91 is situated on the surface of the patient's chest (not shown).

FIG. 10 shows the same arrangement as a coronal cross-section through the patient's heart. The blood-filled cavity inside the left ventricle is surrounded by the annular cross-section through the ventricle walls 900.

Sequential pulses are transmitted from the probe 91 along seven separate transmit beam paths 94a-94g. Any number of transmit beams may be used. These paths are arranged between them to cover the whole perimeter of the ventricle in coronal cross-section, and to illuminate the full thickness of the ventricle wall in the transverse plane, for at least a portion of the ventricle. The angle and shape of these transmit paths 94a-94g is determined by a processor in the scanning apparatus, based on human operator input or analysis of earlier received reflections, for example using image-processing techniques to identify the left ventricle from a 3D imaging scan.

For each transmit beam, a number of curved receive beams 92a, 92a′; 92b, 92b′, etc. are steered within the wall of the ventricle, conforming to its contours. Each transmit beam may support any number of receive beams along different paths: e.g. one, two (as shown here), three, more than ten, or more than 100 receive beams.

Data obtained from along the receive beams 92a, 92a′, 92b, 92b′, etc. may be used for imaging, motion analysis, strain analysis, or with any other appropriate technique. The results of scanning according to the invention may be shown on a display screen, for example as illustrated in FIG. 8, or stored in memory, or communicated over a network.

Although some of these embodiments have been described as steering the focal point of a receive beam along a curved path by adjusting a beam angle in real time (e.g. by dynamically changing delays applied to the signals from each microphone while energy reflected from the transmit pulse is being received), in other variants the tracing of the receive beam along a non-straight path can be accomplished during a post-processing step; e.g. after all the reflected energy from the transmit pulse has been received.

In such arrangements, which may otherwise be similar to those embodiments already described above, the signals from each microphone are captured; e.g. recorded in a computer memory. After reflected energy has been received for a given transmit pulse, the reflections from along a curved path can be extracted by applying suitable delaying-and-summing to the recorded signals, or by any other appropriate beam-forming method. This extraction might happen while reflections from a subsequent transmit pulse are still being captured; e.g. one, two or more transmit pulses later, or may take place much later; e.g. hours or days after the signals were captured.

An advantage of such deferred processing is that the curved paths can be defined after the patient has left the scanning room; e.g. by a doctor a few days later. A disadvantage is that information lost during the capture processing (e.g. due to a limited sampling rate) can mean that the spatial resolution that can be realised along the curved path may be lower than if the delays are adjusted in real-time, which could potentially be done at a higher resolution.

1. A method of acquiring information about a structure within a body, comprising:
providing data defining a non-straight line, at least a part of which corresponds to the structure;
transmitting an ultrasound transmit signal into the body;
receiving reflections of the transmit signal;
processing the received reflections so as to trace a focal point of a receive beam along a path of the non-straight line; and
using reflections of the transmit signal from along the non-straight line to acquire information about the structure.

2. A method as claimed in claim 1 comprising steering the receive beam by delay-and-sum beamforming.

3. A method as claimed in claim 1 wherein the received reflections are processed so as to trace the focal point along the path in real-time.

4. A method as claimed in claim 1 comprising processing received reflections to determine Doppler shift information relating to the structure at one or more points along the non-straight line.

5. A method as claimed in claim 1 comprising using reflections of the transmit signal from along the non-straight line to produce pulse wave Doppler spectrums.

6. A method as claimed in claim 1 comprising determining velocity or strain information relating to the structure at one or more points along the non-straight line.

7. A method as claimed in claim 1 wherein the non-straight line is non-planar.

8. A method as claimed in claim 1 comprising providing data defining a plurality of non-straight lines, and using reflections of the transmit signal from along the plurality of non-straight lines to acquire information about the structure.

9. A method as claimed in claim 8 wherein the plurality of lines do not all lie in a common plane.

10. A method as claimed in claim 8 comprising transmitting a plurality of transmit beams, each transmit beam corresponding to one or more of the non-straight lines.

11. A method as claimed in claim 1 wherein the body is a human or animal body.

12. A method as claimed in claim 11 wherein the structure is subcutaneous.

13. A method as claimed in claim 1 wherein the structure is the outer wall of the left ventricle of a heart.

14. A method as claimed in claim 1 comprising performing an imaging scan of a heart during one heartbeat cycle, and acquiring data about the heart from along one or more non-straight lines during one or more subsequent heartbeats.

15. A method as claimed in claim 1 wherein the non-straight line corresponds to a wall of a left ventricle of a human or animal heart, and comprising using reflections of the transmit signal from along the line to acquire information about a propagation of a shear wave caused by a closing of an aortic valve of the heart.

16. A method as claimed in claim 15 comprising determining information relating to a time taken between the aortic valve closing and a corresponding shear wave reaching a point in the heart along the non-straight line.

17. A method as claimed in claim 15 wherein the line corresponds to a wall of the left ventricle other than a septal wall, and further comprising providing data defining a second non-straight line corresponding to the septal wall and determining information relating to a time delay between an event related to a closing of aortic value detected from a basal septum along the second line and an event related to a closing of the aortic valve detected from another location along the first line.

18. A method as claimed in claim 1 comprising displaying an image of at least a part of the structure.

19. A method as claimed in claim 1 comprising displaying a representation of the non-straight line on a display screen.

20. A method as claimed in claim 1 comprising displaying on a display screen a representation of an area or volume within the structure from which reflections will be processed.

21. A method as claimed in claim 1 comprising using a theoretical model of the structure to provide the data defining the non-straight line.

22. A method as claimed in claim 21 comprising displaying a representation of the model on a display screen.

23. A method as claimed in claim 21 comprising selecting or calibrating the model based on input provided by an operator.

24. A method as claimed in claim 21 comprising processing data acquired by processing ultrasound reflections to select or calibrate the model.

25. A method as claimed in claim 21 comprising iteratively performing the steps of (i) processing ultrasound reflections to acquire information about the structure; (ii) calibrating the model based on said information; (iii) using the model to determine the path of the non-straight line; and (iv) tracing the focal point of a receive beam along said path.

26. A method as claimed in claim 21 comprising determining the path of the non-straight line by applying a criterion or filter to data obtained from ultrasound reflections from the structure.

27. A method as claimed in claim 26 wherein the criterion or filter identifies a tissue boundary.

28. A method as claimed in claim 1 comprising updating a definition of the non-straight line to maintain a correspondence between the line and the structure.

29. A method as claimed in claim 28 comprising transmitting a plurality of transmit signals and updating the definition of the non-straight line one or more times between transmit signals.

30. A method as claimed in claim 1 wherein the non-straight line lies along or spaced away from a boundary surface between the structure and an adjacent region.

31. A method as claimed in claim 1 comprising processing the received reflections so as to trace the focal point along the path of the non-straight line at approximately half the speed of sound.

32. A method as claimed in claim 1 wherein the transmit signal is a pulse.

33. A method as claimed in claim 1 comprising directing energy from the transmit signal so as to encompass all of a part of the curved line which corresponds to the structure.

34. A method as claimed in claim 1 comprising transmitting the transmit signal before the data defining the non-straight line have been provided.

35. A method as claimed in claim 1 comprising providing data that define a transmit path, wherein energy from the transmit signal is arranged to travel at least along this transmit path.

36. Ultrasound scanning apparatus for acquiring information about a structure within a body, comprising:
an arrangement for providing data defining a non-straight line, at least a part of which corresponds to the structure;
a transmitter arrangement arranged to transmit an ultrasound transmit signal into the body;
a receiver arrangement arranged to receive reflections of the transmit signal;
processor configured to:
process the received reflections so as to trace a focal point of a receive beam along a path of the non-straight line; and
use reflections of the transmit signal from along the non-straight line to acquire information about the structure.

37. An ultrasound scanning apparatus as claimed in claim 36 wherein the receiver arrangement comprises an array of receiver elements.

38. An ultrasound scanning apparatus as claimed in claim 36 comprising an input arrangement allowing an operator to input data defining the non-straight line.

39. A carrier bearing software comprising instructions for configuring a processor to carry out the steps of:
processing received signals from reflections of an ultrasound transmit signal transmitted into a body, so as to trace a focal point of a receive beam along a path of a non-straight line, at least a part of which corresponds to a structure within the body; and
using reflections of the transmit signal from along the non-straight line to acquire information about the structure.

40. (canceled)

41. (canceled)

42. (canceled)

43. (canceled)

44. (canceled)

45. (canceled)

46. (canceled)

47. (canceled)

48. (canceled)
